(19) United States
(12) Patent Application Publication
(10) Pub. No.: US 2004/0157798 A1
(43) Pub. Date: Aug. 12, 2004

(54) Drug for use in bone grafting

(76) Inventor: David Graham Little, Westmead (AU)

Correspondence Address: BOZICEVIC, FIELD & FRANCIS LLP, 200 MIDDLEFIELD RD, SUITE 200, MENLO PARK, CA, 94025, US

(21) Appl. No.: 10/474,072

(22) PCT Filed: Mar. 28, 2002
(86) PCT No.: PCT/AU02/00412

(30) Foreign Application Priority Data
Apr. 3, 2001 (AU) PR 4187
Dec. 17, 2001 (AU) PR 9613

(51) Int. Cl. A61K 31/663; A61K 31/675

(52) U.S. Cl. 514/89; 514/102

(57) ABSTRACT

A drug and method of bone grafting which improves the osteoinductive and/or osteoconductive potential of a bone graft, bone graft substitute or extenders. The drug is selected from the group consisting of bisphosphonates which may be administered to a subject either prior to, during or after a bone grafting procedure.

### FIELD OF THE INVENTION

The present invention relates to a drug for use in bone healing. More specifically, the present invention relates to the use of bisphosphonates to increase the osteoinductive and/or osteoconductive potential of bone grafts, bone marrow and bone graft substitutes or extenders.

### BACKGROUND OF THE INVENTION

Bone grafting is a standard technique in orthopaedic surgery, plastic surgery and neurosurgery. The object of bone grafting is to enable healing of bony defects, either to restore bony integrity or create bone bridging between bones such as in spinal or other arthrodeses.

The need to treat a patient with a bone defect may present in a variety of orthopaedic situations. Up to 10% of the 6 million fractures occurring annually in the USA heal with difficulty. In many cases this may result in delayed union or non-union of a fracture resulting in a requirement for bone grafting.

Spinal arthrodesis (spinal fusion) is a common technique requiring bone grafting for which ample autogenous bone graft (bone graft obtained from the patient him/herself) is sometimes lacking. In instances where the autogenous bone graft is lacking, bone graft substitutes or extenders must be used. It is estimated that 500,000 spinal fusions are performed in the USA annually.

Spinal fusion is also enhanced in some situations by the use of porous interbody cages which are usually filled with bone graft or a bone graft substitute or extender. This facilitates the use of less bone graft. Furthermore, the cage takes the load while the bone heals.

Further indications for bone grafting include destruction of bone by tumour or bone cyst formation, the surgical removal of tumorous bone, osteomyelitis, bone defects around arthritic joints (geodes), bone implants, joint replacement prostheses and dental prostheses.

Autogenous bone grafting remains the preferred standard procedure. The procedure involves the use of small amounts of the patient's bone available at the operative site or from a donor site such as the ilium. However, while such a technique is widely accepted, morbidity related to the donor site at the iliac crest has been estimated at as high as 30% and is quite painful in most cases.

In light of the pain associated with the use of autogenous bone graft, there has been a move to replace autogenous bone with bone graft substitutes and synthetic extenders. In this regard, bone graft substitutes are considered to include any physical material other than autogenous bone graft used with the intention of increasing bone formation or bone healing in vivo.

A graft extender is similar to a graft substitute, and may in fact be identical in composition to a substitute. An extender may be used in conjunction with autogenous graft to make the graft “go further”.

Examples of bone graft substitutes and extenders include, but are not limited to, calcium hydroxyapatite (Pro-Osteon®, Pyrost®), calcium sulphate (Osteoset®, Bone Plast™, Jax™) porous tricalcium phosphate (Vitoss™)and Bioglass® (a combination of silicon, sodium, calcium and phosphorus). Pro Osteon® 200R is a resorbable, osteoconductive matrix consisting of hydroxyapatite and calcium carbonate. Tricalcium Phosphate cement (Norian ® SRS™, alpha-BSM™) is also available in an injectable form. Most of these products fully resorb fully in vivo, which is a preferred feature of the present invention but in some cases, the resorption of the calcium complex is incomplete. In such cases, the osseointegrated product is typically biocompatible such that this has no clinical consequence.

Recent developments involve the- inclusion of various bone promoting growth factors in a delivery medium. Demineralised bone matrix in a gel, putty, sheet or other forms (Grafton®), DynaGraft®, Osteofil®, AlloGro® etc) are available. The demineralised bone still contains some of the mediators implicated in bone healing and is typically taken from allograft (human bone from a cadaver processed such that infective agents are eliminated) or xenograft (bone or other tissue from an animal source) which has had the calcium removed. This process often leaves many of the gene products. (proteins) known to upregulate bone formation in the graft substitute. These products are osteoinductive and may also be osteoconductive, that is, they provide a framework or scaffold on which cells and primitive tissue can attach and begin the process of new bone formation.

However, there remain concerns about disease transmission or allergic reactions associated with such materials. To address this, autogenous blood products containing autogenous growth factors (AGF™) such as platelet derived growth factors (PDGF) and other factors in the ‘buffy coat’ are sometimes used in addition to bone grafting or bone graft substitutes.

Alternatively, refined human gene products such as Bone Morphogenetic Protein 7 (OP-1), Bone Morphogenetic Protein 2 and other Bone Morphogenetic proteins (BMPs) are available, but these are expensive osteoinductive agents. Other products using transforming growth factor beta (TGF-β), Fibroblast Growth Factor (FGF) and Insulin-like Growth factor (IGF) are in development.

As autogenous bone marrow contains osteoprogenitor stem cells, it is often used to augment the osteoinductive cellular response to bone grafting. In an extension of this concept, osteoprogenitor stem cells can be identified from the harvested marrow and cultured in vitro. A large number of autogenous stem cells may then be transferred back into the operative site in a process known as stem cell transfer. The bone marrow and bone forming cells may be administered simply as harvested or, alternatively, admixed with a carrier.

The variety of methods available to enable bone healing to occur in clinical situations underscores the fact that these operations are not always successful.

Many of the abovementioned options have serious limitations. In general, the simpler products, while less expensive, are less effective bone graft substitutes when compared to the more complicated bioactive substances containing stem cells or bone morphogenetic proteins. Thus, while simple readily available methods are somewhat limited by their effectiveness, more effective methods are limited by cost.

Bisphosphonate drugs were thought to have their main clinical use in preventing bone resorption. Recent studies, however, suggest that bisphosphonates in certain dose ranges have properties conducive to bone formation, whilst minimally interfering with resorption and remodelling of a bone.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In a first aspect, the present invention consists in a drug selected from the group consisting of at least one bisphosphonate when used for treating a patient requiring bone grafting.

In a second aspect, the present invention consists in a drug selected from the group consisting of at least one bisphosphonate when used for improving the osteoinductive and/or osteoconductive potential of a bone graft.

In a third aspect, the present invention consists in a drug selected from the group consisting of at least one bisphosphonate when used for improving the ostebinductive and/or osteoconductive potential of bone graft substitutes or extenders.

In a fourth aspect, the present invention consists in a drug selected from the group consisting of at least one bisphosphonate when used for improving the osteoinductive and/or osteoconductive potential of bone graft substitutes or extenders when said bone graft substitutes or extenders are used in combination with a bone graft or with each other.

In a fifth aspect, the invention consists in a method of performing a bone graft procedure including the step of administering to a patient a therapeutically effective amount of a drug selected from the group consisting of at least one bisphosphonate.

In a sixth aspect, the invention consists in a method of performing bone grafting in a patient, said method including the steps of:

(a) harvesting autogenous bone graft from a donor site of the patient;

(b) delivering said harvested autogenous bone graft to a graft site;

(c) administering a drug selected from the group consisting of at least one bisphosphonate. to the patient to increase the osteoinductive and/or osteoconductive potential of the autogenous bone graft.

In a seventh aspect, the present invention consists in a method of performing bone grafting in a patient, said method including the steps of:

(a) delivering a bone graft, bone graft substitute, an extender or a combination thereof to a graft site; and

(b) administering a drug selected from at least one bisphosphonate to the patient to increase the osteoinductive and/or osteoconductive potential of the bone graft, bone graft substitute or the extender.

In an eighth aspect, the present invention consists in a drug selected from the group consisting of at least one bisphosphonate when used for improving the osteoinductive potential of autogenous bone marrow.

The bone marrow may be harvested from the patient for implantation into the graft site. The bone marrow may be admixed with a therapeutically effective amount of at least one bisphosphonate prior to implantation.

The bone marrow and bisphosphonate may further be mixed with a resorbable carrier of the type described above.

The bone marrow may also be admixed with autogenous bone graft, bone graft substitutes or extenders in addition to the bisphosphonate before implantation into the graft site.

In a further embodiment, the bone marrow is processed following its harvest and the bone stem cells in the marrow caused to multiply. The stem cells derived from the bone marrow are then implanted into the graft site. Stem cells may also be harvested from some other site, e.g. muscle or fat.

The bone marrow or cells, in addition to being admixed with a therapeutically effective amount of bisphosphonate may further be admixed with nutrients required by cells involved in bone formation or other cells.

In a ninth aspect, the present invention consists in a method of performing bone grafting in a patient, said method including the steps of:

(a) harvesting bone marrow from a patient;

(b) delivering said harvested bone marrow to a graft site;

(c) administering a drug selected from the group consisting of at least one bisphosphonate to the patient to increase the osteoinductive potential of the implanted bone marrow.

In the embodiment of the invention wherein bone graft is used in the grafting procedure, the bone graft is typically autogenous bone graft, that is, small pieces of bone harvested from the patient subject of the bone grafting procedure. This bone may be harvested from the operative site or percutaneously from a donor site such as the ilium. Alternatively, an open approach to the ilium or other donor site may be used to harvest the graft

In another embodiment, the bone graft may include allograft (human bone from a cadaver which is processed such as to remove any infectious agents) or xenograft (bone from an animal source).

Where an extender is used, said extender is preferably any material other than autogenous bone graft which is adapted to increase the amount of graft material for implantation or injection into a graft site. Preferably synthetic calcium complexes are used as extenders. Examples of extenders include, but are not limited to, calcium hydroxyapatite (Pro-Osteon®, Pyrost®), calcium sulphate (Osteoset® and Bone Plast™) porous tricalcium phosphate (Vitos ™)and Bioglass® (a combination of silicon, sodium, calcium and phosphorus). Pro Osteon® 200R is a resorbable, osteoconductive matrix consisting of hydroxyapatite and calcium carbonate. Tricalcium Phosphate cement (Norian ®) is also available in an injectable form. Most of these products fully resorb fully in vivo, which is a preferred feature of the present invention but in some cases, the resorption of the calcium complex is incomplete. In such cases, the osseointegrated product is typically biocompatible such that this has no clinical consequence.

The extender may be mixed with other pharmacologically active substances such as antibiotics.

In another embodiment, the bone graft substitute is preferably any material other than autogenous bone graft. Examples of suitable bone graft substitutes include, but are not limited to, calcium hydroxyapatite (Pro-Osteon ®, Pyrost®), calcium sulphate (Osteoset®, Bone Plast™ and JAX™), porous tricalcium phosphate (Vitoss™), Bioglass® (a combination of consisting of silicon, sodium, calcium and phosphorus), Pro Osteon® 200R (resorbable, osteoconductive matrix consisting of hydroxyapatite and calcium carbonate) and Monocalcium and tricalcium phosphate, calcium carbonate and liquid sodium phosphate cement (Norian® SRS™), or other injectable calcium phosphate substitute (eg Alpha-BSM™). Most of these products resorb fully in vivo, which is a preferred feature of the present invention. In some cases, however, the resorption of the calcium complex is incomplete. In such cases, the osseointegrated product is preferably biocompatible such that this has no clinical consequence. In this embodiment other pharmacologically active substances may be included with the bone graft substitute such as antibiotics.

The bone graft substitutes or extenders may also contain gene products known to be implicated in bone healing. Examples of suitable gene products include, but are not limited to, Bone Morphogenetic Protein 7 (OP1), BMP-2 and -4 and -6, other bone morphogenetic proteins, transforming growth factor beta, fibroblast growth factor, insulin-like growth factor, osteocalcin, or other known biologically active proteins, polypeptides, or gene products.

In one embodiment, the at least one bisphosphonate is admixed locally at a graft site with the bone graft or the bone graft substitute or the extender or a combination thereof.

However, it is preferred that the at least one bisphosphonate is admixed with the bone graft or bone graft substitute or extender prior to a bone grafting procedure and the admixture administered to the graft site thereafter.

Particularly, it is envisaged that the bone graft substitute or extender is manufactured to include a therapeutically effective amount of the at least one bisphosphonate.

It is a particularly preferred feature of the present invention that the amount of bisphosphonate in the manufactured bone graft substitute or extender is such that new host bone formation is enhanced without substantially interfering with the gradual resorption of the said bone graft substitute or extender (or when used in conjunction with bone graft, the bone graft), such that the bone graft substitute, extender or bone graft together with any carrier medium is completely resorbed and replaced by normal remodelled host bone in the long term.

The bone graft, the bone graft substitute or the extender or a combination thereof may be administered to the graft site as a first step and the at least one bisphosphonate administered systemically to the patient thereafter. The at least one bisphosphonate may also be directly administered to a graft site following administration of the bone graft, the bone graft substitute or the extender or a combination thereof.

Alternatively, the at least one bisphosphonate may be delivered systemically before the bone graft, the bone graft substitute or the extender is administered to the graft site. Furthermore, the at least one bisphosphonate may be given intra-operatively such that the bone graft, the bone graft substitute or the extender or a combination thereof may be administered to the graft site simultaneously with systemic bisphosphonate administration.

The at least one bisphosphonate may be administered intravenously although it is also envisaged that the bisphosphonate may be administered orally. Further, the at least one bisphosphonate may be administered subcutaneously, intramuscularly, transdermally, topically or by any other parenteral route by which it can produce its systemic effect. Combinations of these routes are also envisaged.

In a preferred embodiment of the invention, the bisphosphonate is zoledronic acid (zoledronate) {1-hydroxy-2-[(1H-imidazol-1-yl)ethylidine]bisphosphonic acid}. Alternatively, the bisphosphonate may be, but is not limited to, any one of the following:

pamidronate {3-amino-1-hydroxypropylidene bisphosphonic acid};

alendronate {4-amino-1-hydroxybutylidene bisphosphonic acid};

etidronate {1-hydroxyethylidene bisphosphonic acid};

clodronate {dichloromethylene bisphosphonic acid};

risedronate {2-(3-pyridinyl)-1-hydroxyethylidene bisphosphonic acid};

tiludronate {chloro-4-phenylthiomethylidene bisphosphonic acid};

ibandronate {1-hydroxy-3(methylpentylamino)-propylidene bisphosphonic acid};

incadronate:{cycloheptyl-amino-methylene bisphosphonic acid};

minodronate:{[1-hydroxy-2-(imidazo[1,2-a]pyridin-3-yl)ethylidene]bisphosphonic acid};

olpadronate:{(3-dimethylamino-1-hydroxypropylidene) bisphosphonic acid};

neridronate (6-amino-1-hydroxyhexylidene-1,1-bisphosphonic acid);

EB-1053:1-hydroxy-3-(1-pyrrolidinyl)-propylidene-1,1-bisphosphonic acid;

or any other therapeutically effective bisphosphonate or pharmaceutically acceptable salts or esters thereof.

Preferably, zoledronic acid (zoledronate) is administered parenterally at 0.01 to 0.5 mg/kg body weight per dose.

In a further embodiment, other bisphosphonates may be administered parenterally as follows:

pamidronate at 0.01 to 3.0 mg/kg body weight per dose;

ibandronate at 0.01 to 0.5 mg/kg body weight per dose;

risedronate at 0.01 to 0.5 mg/kg body weight per dose;

alendronate at 0.01 to 5.0 mg/kg body weight per dose;

clodronate at 0.01 to 20 mg/kg body weight per dose;

etidronate at 0.01 to 20 mg/kg body weight per dose;

tiludronate at 0.01 to 5.0 mg/kg body weight per dose;

incadronate at 0.01 to 5.0 mg/kg body weight per dose;

minodronate at 0.01 to 0.5 mg/kg body weight per dose;

olpadronate at 0.01 to 0.5 mg/kg body weight per dose;

neridronate at 0.01 to 5.0 mg/kg body weight per dose;

EB-153 at 0.01 to 5.0 mg/kg body weight per dose.

It is further envisaged that the following bisphosphonates may be administered orally:

zoledronic acid (zoledronate), pamidronate, risedronate, ibandronate, minodronate, alendronate, tiludronate, incadronate, olpadronate, neridronate, clodronate, etidronate, tiludronate or EB-1-53;

wherein said bisphosphonates are administered in doses from 0.01 to 5 mg/kg body weight daily, or the equivalent doses given second daily or weekly.

In another embodiment, the bisphosphonate is initially administered parenterally, followed by oral administration of the bisphosphonate wherein the oral bisphosphonate is administered in a dose from 0.01 to 5 mg/kg body weight daily, or the equivalent dose given second daily or weekly.

In one embodiment of the invention, zoledronic acid (zoledronate) is admixed with bone graft or a bone graft substitute or an extender or combinations thereof at 0.0001 to 0.5 mg/kg body weight per dose. The zoledronic acid (zoledronate) may be directly applied at the time of surgery or have been incorporated in the manufacturing process of the bone graft substitute or extender.

Other bisphosphonates may be admixed with bone graft or a bone graft substitute or an extender or combinations thereof as follows:

pamidronate, preferably administered at 0.0001 to 3.0 mg/kg body weight per dose

ibandronate (ibandronic acid) preferably administered at 0.0001 to 0.5 mg/kg body weight per dose;

risedronate preferably administered at 0.0001 to 0.5 mg/kg body weight perdose;

alendronate, preferably administered at 0.0001 to 5.0 mg/kg body weight per dose;

clodronate, preferably administered at 0.0001 to 20 mg/kg body weight per dose;

etidronate, preferably administered at 0.0001 to 20 mg/kg body weight per dose;

tiludronate, preferably administered at 0.0001 to 5.0 mg/kg body weight per dose;

incadronate, preferably administered at 0.0001 to 5.0 mg/kg body weight per dose;

minodronate, preferably administered at 0.0001 to 0.5 mg/kg body weight per dose;

olpadronate, preferably administered at 0.0001 to 0.5 mg/kg body weight per dose;

neridronate, preferably administered at 0.01 to 5.0 mg/kg body weight per dose;

EB-153 0.0001, preferably administered at 5.0 mg/kg body weight per dose.

These bisphosphonates may be directly applied at the time of surgery or may be incorporated in the manufacturing process of the bone graft substitute or extender.

Autogenous bone graft may be harvested percutaneously from a donor site of the patient by way of a bone trocar or other percutaneous bone harvesting device. Alternatively, small amounts of autogenous bone graft may be readily harvested from the operative site. Following either means of harvest, the autogenous bone graft may be mixed with a therapeutically effective amount of bisphosphonate and the mixture subsequently implanted or injected into the graft site to enhance host bone formation at the graft site while still allowing resorption of the autogenous bone graft by the host such that normal remodelled bone fills the graft site in the long term.

In another embodiment, bone graft, a bone graft substitute, an extender or a combination thereof, in addition to being admixed with an effective amount of bisphosphonate may be further mixed with a carrier medium such as collagen, gelatine, glycerol, resin, polyglycolic acid, polylactic acid, or any other fully resorbable biocompatible medium, or a combination thereof, either in the form of injectable liquid, gel, putty or cement or in the form of mouldable liquid, cement, putty, gel, flexible sheets, mesh or sponge or other readily applicable method.

The carrier may contain nutrients required by cells involved in bone formation or other cells and is preferably resorbable. The carrier may further contain gene products known to be implicated in bone healing. Examples of suitable gene products include, but are not limited to, Bone Morphogenetic Protein 7 (OP-1), BMP-2,-4 or 6 or other bone morphogenetic proteins, transforming growth factor beta, fibroblast growth factor, insulin-like growth factor, osteocalcin, or other known biologically active proteins, polypeptides, or gene products.

In a further embodiment a carrier medium is used alone with a bisphosphonate. In this embodiment, the carrier may be, for example, collagen, gelatin or a resorbable water based gel.

An indication for bone grafting in a patient may be bone loss resulting from trauma including fracture, delayed union or non-union of bone, or bone loss due to infection following open fracture or open treatment of a closed fracture. Alternatively, the patient may have a bone defect unrelated to trauma, including, but not limited to tumour or cyst formation, osteolysis from metabolic disorders such as hyperparathyroidism or renal failure, osteomyelitis, or surgical removal of bone.

In a further embodiment, the patient has bone defects in the maxilla or the mandible and is undergoing dental or plastic surgical reconstruction.

In another embodiment the patient has a skull defect.

In a further embodiment, the patient has undergone an osteotomy and bone grafting is required to assist bony union.

The patient may be undergoing an arthrodesis of a joint or joints. In this regard, the patient may be undergoing a spinal arthrodesis (spinal fusion) operation, either posterior or anterior, with or without the use of internal fixation devices, including interbody spinal fusion cages.

In one embodiment, the cages contain autogenous graft, allograft or xenograft.

In another embodiment, the interbody spinal fusion cage may be coated with a bisphosphonate, or mixture of bisphosphonate and any calcium containing compound such as, but not limited to, calcium sulphate, tricalcium phosphate, hydroxyapatite. The cage may alternatively be filled with bisphosphonate particles or particles of bone graft substitute or extender admixed with a therapeutically effective amount of bisphosphonate. The cage may alternatively also contain known mediators of bone formation such as BMP's or TGF-beta or other growth factor or cytokine. Further, the patient may receive bisphosphonates systemically, and the cage may be empty or contain any combination of autogenous bone graft, bone graft substitute or extender, or known mediators of bone formation such as BMP's or TGF-beta or other suitable growth factors or cytokines

Further, the patient may be suffering from congenital pseudarthrosis of the tibia, fibula or other bone, or a related condition. In this embodiment the bisphosphonate acts to improve the osteoinductive and/or osteoconductive potential of a graft and reverse the primary disorder of local cell function towards osteogenesis. Continued bisphosphonate therapy to contain the pathologic process underlying the disorder and prevent re-fracture and re-formation of a pseudarthrosis may be added after union is achieved.

In another embodiment, the patient may be undergoing revision or complicated primary joint arthroplasty, where bone grafting is required. In this embodiment, it is envisaged that the grafts are not of the large structural graft type typically used to support the arthroplasty immediately, but rather morsellised bone grafts collected from reamings locally or impaction bone grafts (morsellised allografts) of the femur or pelvis. However in some circumstances large structural grafts, or a combination of structural and morsellised grafts may be preferred when used with a therapeutic amount of a bisphosphonate such that the bone is eventually resorbed and replaced with normal host remodelled bone in the long term.

In another embodiment the patient may require the use of bone graft, bone graft substitutes or extenders to fill an alveolar bone defect around a dental prostheses.

In a tenth aspect, the present invention consists in a drug selected from the group consisting of bisphosphonates when used for treating a patient requiring a spinal or other joint arthrodesis.

In one embodiment of the tenth aspect, the drug is used to increase the osteoinductive and/or osteoconductive potential of small particles of graft typically obtained locally at a selected area during surgery. The increase in osteoinductive and/or osteoconductive potential of the graft increases the fusion rate of the arthrodesis.

The bisphosphonate may be administered to the patient parenterally at or near the time of the surgery. This may be followed by a second parenteral dose of bisphosphonate administered from between two to six weeks after the initial surgery.

In a further embodiment, the initial parenteral dose of bisphosphonate may be followed by administration of oral bisphosphonates in a daily or second daily or weekly regimen commencing about one to three months after the initial dose for a period of about two months or until sufficient new bone (as assessed by the treating doctor) has been produced.

In a further embodiment, further to the abraded graft and the bisphosphonate, it is envisaged that further bone graft, bone marrow, bone graft substitute or extender, or carrier or combinations thereof may be administered to the patient to increase the fusion rate in an arthodesis.

In an eleventh aspect, the present invention consists in a drug selected from the group consisting of at least one bisphosphonate when used for improving the osteoinductive and/or osteoconductive potential of bone graft, bone graft substitutes or extenders wherein said bone graft, bone graft substitutes or extenders are held within a cage for interbody spinal fusion.

The cage may be made of titanium or any other biocompatible metal or alloy, or of a resorbable polymer or calcium containing complex. The cage may further be coated directly with the bisphosphonate or the bisphosphonate may be given systemically to encourage interbody fusion. The bisphosphonate may be administered either alone or in combination with bone graft, allograft, xenograft, bone graft substitutes or extenders, mediators of bone formation or any combination of the above.

In a twelfth aspect, the present invention consists in a device for performing a bone grafting procedure, the device including a first receiving means for receiving any one of bone graft, bone marrow, bone graft substitutes, extenders or carriers or a combination thereof and at least a second receiving means for receiving a drug selected from the group consisting of at least one bisphosphonate, the device further including a mixing means to allow mixing of said drug with said bone graft, bone marrow, bone graft substitutes, extenders, carriers or combination thereof.

The device of the twelfth aspect may further include a means to allow the mixture to be directly injected or implanted into a graft site of a patient. Alternatively, the mixture may be removed from the device and delivered to the graft site as a separate step.

Throughout this specification, the term “a therapeutically effective amount of a bisphosphonate” is defined as that which will increase the osteoinductive and/or osteoconductive potential of the bone graft, bone graft substitute or extender such that bone formation is increased in both amount and mineralisation, without interfering with the process of eventual resorption of the bone graft, substitute or extender, such that the bone graft, substitute or extender is completely replaced by normal remodelled host bone in the long term. In the case of calcium containing synthetic bone graft substitutes, the resorption of some of the calcium complex may be incomplete due to its own properties, but the osseointegrated product is biocompatible such that this has no clinical consequence.

### BRIEF DESCRIPTION OF THE DRAWINGS

By way of example only, a preferred embodiment of the invention is described with reference to the accompanying figures:

FIG. 1 is a generic formula for one class of bisphosphonates;

FIG. 2 depicts a device for percutaneous bone graft harvesting;

FIG. 3 is a CT scan of a calcium sulphate cylinder;

FIG. 4 is a set of CT scans at four weeks; and

FIG. 5 is a set of CT scans at 6 weeks.

### PREFERRED EMBODIMENT OF THE INVENTION

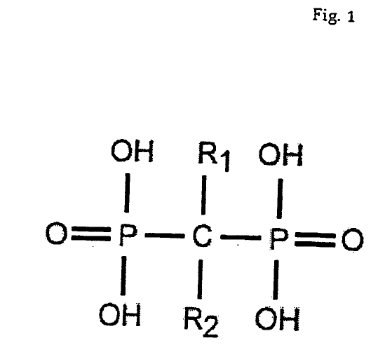

The invention relates to the use of a bisphosphonate with the general formula depicted in FIG. 1 wherein R1 is varied to give binding and solubility properties to the bisphosphonate and R2 is varied to give various potencies and other properties to the bisphosphonate.

The present invention relates to the pharmacological use of bisphbsphonates in bone grafting. In particular, the present inventor has found that bisphosphonates increase the inductive and/or osteoconductive potential of various types of graft components including autogenous bone graft, bone marrow, bone graft substitutes and extenders. Accordingly, when a bisphosphonate is administered to a patient, less graft is required. This is of particular significance in certain embodiments wherein the graft is autogenous bone graft or autogenous bone marrow. As such grafts are harvested from the patient themselves, the harvesting of larger amounts of bone or marrow will cause an undesirable level of pain and discomfort for the patient.

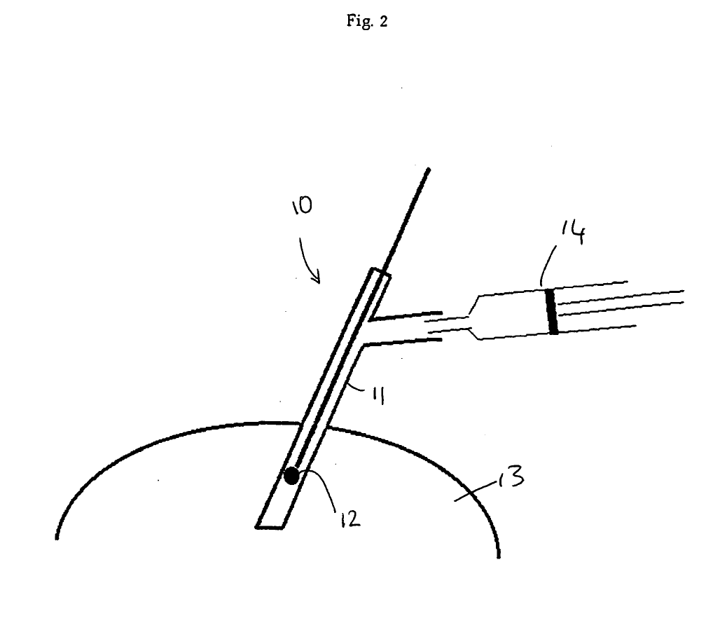

An example of a device which may be used to harvest bone graft from a patient is generally depicted as 10 in FIG. 2 of the accompanying drawings. The device 10 includes a trocar 11, said trocar having a burr or drill 12 positioned within. In the present example, bone graft is forced up from the ilium 13 of a patient and into the body of the trocar 11 as the burr or drill 12 descends. The bone graft may then be removed from the trocar 11 by way of, for example, a syringe 14.

### EXAMPLE

The following example outlines the process by which the effects of bisphosphonates on potentiating new bone formation in a graft may be observed. In this regard, the effects on bone mineralisation and the mechanical properties are observed in a 10 mm tibial defect model in rabbits.

Three groups of animals tested consist of:

Vehicle: Calcium sulphate and vehicle (saline)

Bisphosphonate: Calcium sulphate and vehicle (saline) and two doses of intravenous bisphosphonate.

Local Bisphosphonate: Calcium sulphate and locally administered bisphosphonate.

Bisphosphonates and Bone Healing

The present inventor has previously performed several successful experiments utilising bisphosphonates to improve new bone formation and reduce stress-shielding osteoporosis in a distraction osteogenesis model in the NZW rabbit. The results of this experiment are included in International Application No PCT/AU00/00982, the contents of which are herein incorporated by reference.

Animal Model

The present experiment uses an 8-week-old NZW rabbit model. A 10 mm gap is surgically removed from the tibiae of the rabbits. The bones are held with a M-100 external fixator.

Methods

Preparation of Calcium Sulphate (Osteoset BVF) Cylinders

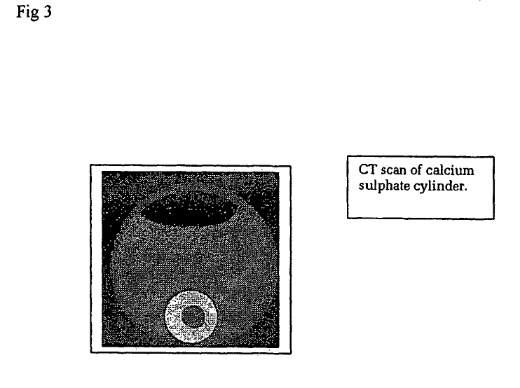

Cylinders of calcium sulphate (Osteoset) were prepared on the day of surgery. Moulds were prepared using a combination of 1.0 ml and 3.0 ml syringes, and the Osteoset poured into the moulds in a sterile environment. The cylinders were removed from the moulds at 12 minutes, and allowed to dry in air for 90 minutes, followed by final drying in a microwave for 30 seconds. Once dry, the cylinders were formed into a standard dimension of 10 mm length, with an external diameter of 9 mm and with a 3 mm diameter internal core. The cylinders weighed 0.80.9 g. The cylinders were stored in sterile environment until required. A CT scan of a calcium sulphate cylinder is shown in FIG. 3.

At the commencement of the operative procedure 0.15 ml of saline, or 0.15 ml of zoledronic acid (0.05 mg/kg) was adsorbed onto a calcium sulphate cylinder, which was then replaced into the sterile container.

Surgery

Surgery was performed in an animal theatre. The rabbits received premedication and sedation (IM Ketamine 15 mg/kg, Xylazine 4 mg/kg) given in combination 10 minutes before surgery. Anaesthesia was achieved using inhaled Halothane 2% and Oxygen 1 L/min.

The operative field was prepared by shaving with clippers, disinfected with povidone iodine 4% w/v in 70% alcohol. The right tibia was exposed sub-periosteally along its length and four 3 mm Orthofix pins inserted. An M-100 monolateral external fixator (Orthofix S.L.R) was applied. A 10 mm piece of mid-diaphyseal tibia was removed using an oscillating saw, along with the central 10 mm of periosteum. A calcium sulphate cylinder was then placed in the defect, and held by slight compression of the external fixator.

The animals were randomised such that at the time of surgery, 24 animals had Osteoset cylinders with saline adsorbed inserted, 12 of whom were given saline-only infusions and 12 of whom were given 0.1 mg/kg zoledronic acid infusion at the time of surgery and again on day 14. A further 12 animals had Osteoset cylinders with 0.15 ml of zoledronic acid (0.05 mg/kg) adsorbed inserted, and given a saline only infusion. Note that in the third leg the zoledronic acid was administered to the site via the cylinder, and not directly.

The operative field was irrigated with Normal Saline and then with a solution containing 600 mg of Benzyl-penicillin to minimise the risk of infection. The wound was then closed in layers with an interrupted dissolvable suture. Buprenorphine 0.05 mg/kg was administered at the end of surgery and again 12 hours post-operatively to all animals. The animals were supplied with rabbit pellet and water ad libitum.

Animals were culled at 28 days (18 animals) and at 42 days (18 animals) with IV pentobarbitone 150 mg/kg.

All animals had radiographic examinations performed at 14 and 28 days, with radiographic examination of the remaining 18 animals at 42 days. CT scans were performed at 28 days (18 animals) and at 42 days (18 animals) using a Stratec XCT Research SA pQCT scanner.

Results

Two four-week controls were excluded—one was culled due to wound problems and one had failure of the fixator. One six-week control had failure of the fixator.

Radiographs showed that the calcium sulphate cylinders were largely dissolved by week 2, and not detectable by week 4. There was a visual trend for improved bone formation at the site of the cylinder in the locally dosed group, but this was difficult to quantify on the plain radiographs, due to variable periosteal bone formation. Quantification was based on the CT scan results.

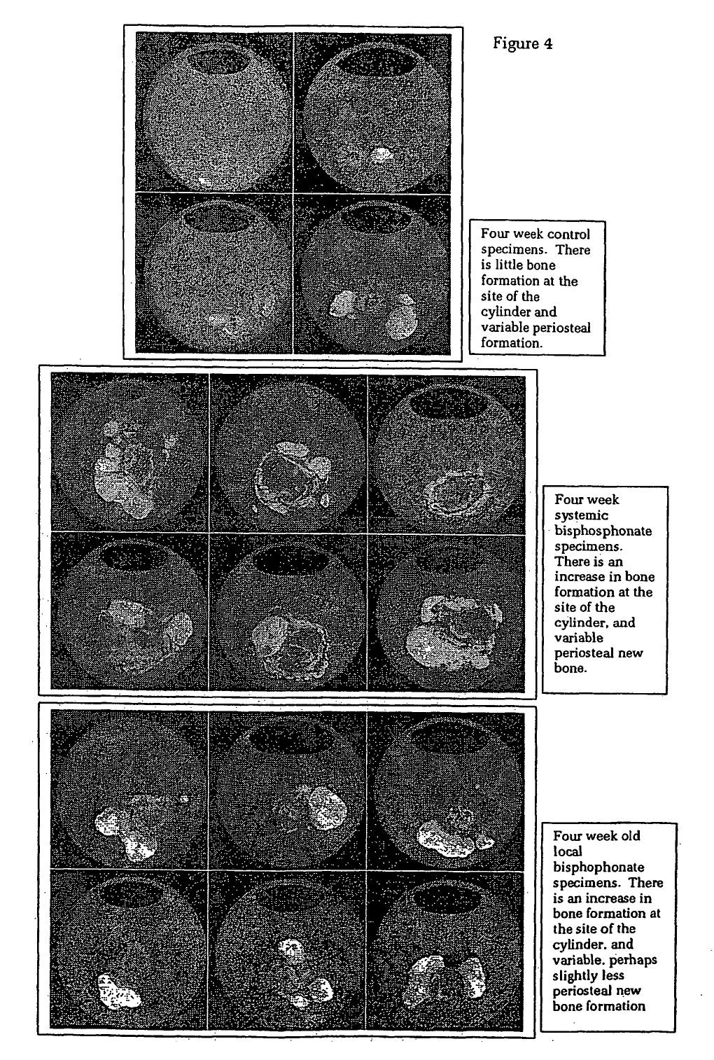

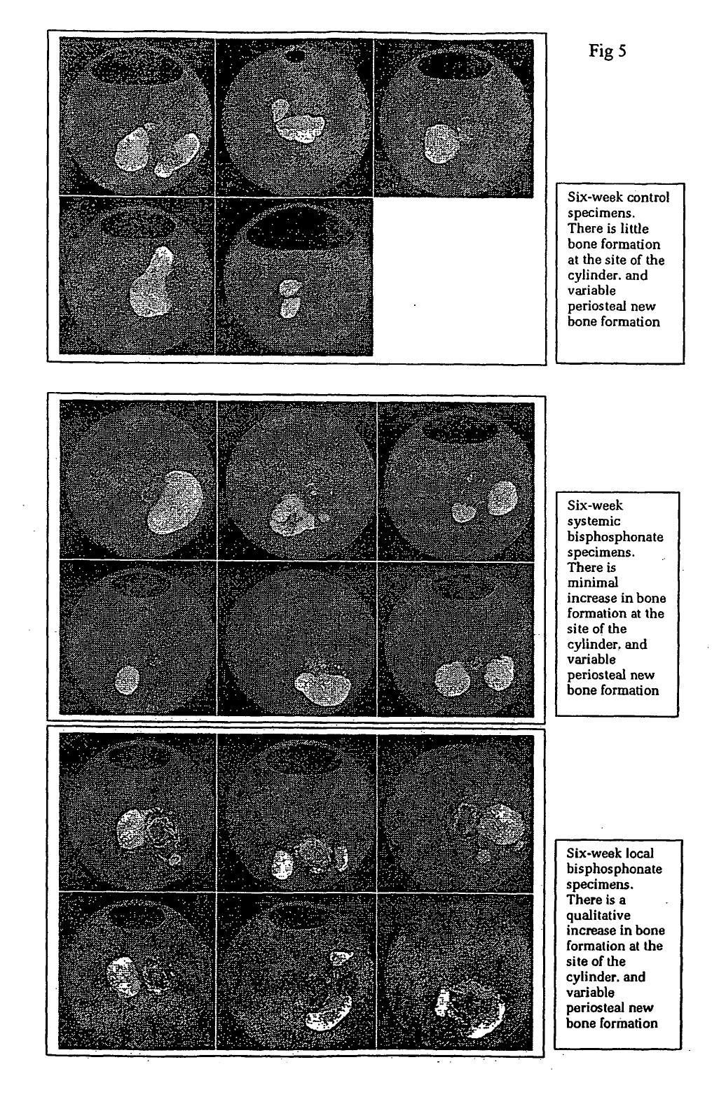

There was a significant increase in bone formation noted in the CT scan slice in the centre of the healing defect at 4 weeks in the treated animals over controls (p<0.05). Bone area was increased 131% and 216% in the IV and Local ZA groups, respectively. Polar moment of inertia was increased by 104% and 316% in the IV and Local ZA groups, respectively. The bone mineral content was likewise significantly increased in the treated groups (p<0.05). At six weeks the cross sectional area was increased by 20% in local bisphosphonate group but this was not significant due to high variability in the periosteal bone formation. The polar moment of inertia was still increased by 40% in the Local ZA group over controls at six weeks. BMC was roughly the same across groups at six weeks. Qualitatively, there was an increase in direct bone formation at the site of the calcium sulphate cylinders, even at six weeks (FIGS. 4 and 5).

| | Control | IV ZA | Local ZA |
|---|---|---|---|
| 4 Weeks | | | |
| Total Bone Area | 31.1 (24.8) | [symbol] | [symbol] |
| (mm²) | | | |
| BMC (mg/mm) | 16.1 (15.6) | [symbol] | [symbol] |
| BMD (mg/cm³) | 462.7 (107.8) | [symbol] | [symbol] |
| Polar Moment | 791.6 (969.5) | [symbol] | [symbol] |
| (mm⁴) | | | |
| 6 Weeks | | | |
| Total Bone Area | 55.8 (22.8) | 58.0 (23.2) | 66.6 (7.9) |
| (mm²) | | | |
| BMC (mg/mm) | 42.1 (15.5) | 45.9 (20.7) | 45.0 (5.4) |
| BMD (mg/cm³) | 771.0 (62.9) | 784.6 (97.7) | 676.2 (49.3) |
| Polar Moment | 954.1 (842.1) | 442.8 (585.2) | 1343.1 (254.7) |
| (mm⁴) | | | |

*Items in BOLD Italic are significantly increased over control.

Discussion

The applicant has previously documented improvements in amount, mineral density and strength of bone in a rabbit distraction osteogenesis model when one or two doses of IV zoledronic acid are given. This experiment documents that local bisphosphonate administration can favourably alter the amount and density of calcium sulphate mediated bone formation in a bone. defect.

There was some concern that the bisphosphonate may interfere with the dissolution of the calcium sulphate, or that the calcium sulphate would interfere with the bisphosphonates pharmacological action. These concerns were disproved, with increases in bone formation in both local and IV groups, but with longer lasting benefit in the local administration group.

The CT results clearly show a benefit of administration of bisphosphonate with a bone graft substitute.

Conclusion

The performance of a calcium sulphate cylinder bone graft substitute in a rabbit tibial defect model can be enhanced by both systemic and local bisphosphonate administration.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A drug selected from the group consisting of at least one bisphosphonate when used for treating a patient requiring bone grafting.

2. A drug selected from the group consisting of at least one bisphosphonate when used for improving the osteoinductive and/or osteoconductive potential of a bone graft.

3. A drug selected from the group consisting of at least one bisphosphonate when used for improving the osteoinductive and/or osteoconductive potential of bone graft substitutes or extenders.

4. A drug selected from the group consisting of at least one bisphosphonate when used for improving the osteoinductive and/or osteoconductive potential of bone graft substitutes or extenders when said bone graft substitutes or extenders are used in combination with a bone graft or with each other.

5. The drug of claim 2 wherein the bone graft is autogenous bone graft.

6. The drug of claim 2 wherein the bone graft is allograft or xenograft.

7. The drug of claim 3 wherein the at least one bisphosphonate is admixed with the bone graft substitute and/or extender during the manufacture of said bone graft substitute and/or extender.

8. The drug of claim 3 wherein the bone graft substitutes or extenders comprise a synthetic calcium complex.

9. The drug of claim 3 wherein the extender and/or the bone graft substitute is fully resorbable within the body of a patient.

10. The drug of any one of claims 1 to 6, 8 or 9 wherein the at least one bisphosphonate is administered intravenously, orally, subcutaneously, intramuscularly, transdermally, or topically to a patient in need of such treatment.

11. The drug of any one of the preceding claims wherein the at least one bisphosphonate is zoledronic acid (zoledronate) or a pharmaceutically acceptable salt or ester thereof.

12. The drug of any one of claims 1 to 10 wherein the at least one bisphosphonate is selected from pamidronate, alendronate, etidronate, clodronate, risedronate, tiludronate, ibandronate, incadronate, minodronate, olpadronate, neridronate or EB-1053 or pharmaceutically acceptable salts or esters thereof.

13. The drug of claim 11 wherein the zoledronic acid (zoledronate) is administered parenterally at 0.01 to 0.5 mg/kg body weight per dose.

14. The drug of claim 11 wherein the zoledronic acid (zoledronate) is admixed with bone graft or a bone graft substitute or an extender or combinations thereof at 0.0001 to 0.5 mg/kg body weight per dose.

15. The drug of claim 3 wherein the bone graft substitute or extender, or a combination thereof, is admixed with a carrier medium selected from collagen, gelatine, glycerol, resin, polyglycolic acid, polylactic acid, or any other fully resorbable biocompatible polymer or a combination thereof either in the form of injectable liquid, gel, putty or cement or in the form of mouldable liquid, cement, putty, gel, flexible sheets, mesh or sponge.

16. The drug of claim 15 wherein the at least one bisphosphonate is admixed with the carrier medium during the manufacture of said carrier medium.

17. The drug of claim 15 or claim 16 wherein the carrier includes gene products selected from Bone Morphogenetic Protein 7 (OP-1), BMP-2 -4 or -6, transforming growth factor beta, fibroblast growth factor, insulin-like growth factor or osteocalcin.

18. Use of a drug selected from the group consisting of at least one bisphosphonate for the manufacture of a medicament for treating a patient undergoing a bone grafting procedure.

19. Use of a drug selected from the group consisting of at least one bisphosphonate for the manufacture of a medicament for improving the osteoinductive and/or osteoconductive potential of a bone graft.

20. Use of a drug selected from the group consisting of at least one bisphosphonate for the manufacture of a medicament for improving the osteoinductive and/or osteoconductive potential of bone graft substitutes or extenders.

21. A drug selected from the group consisting of at least one bisphosphonate when used for improving the osteoinductive potential of autogenous bone marrow.

22. The drug of claim 21 wherein the bone marrow is admixed with the at least one bisphosphonate prior to implantation in a subject.

23. The drug of claim 21 or claim 22 in combination with a resorbable carrier medium selected from collagen, gelatine, glycerol, resin, polyglycolic acid, polylactic acid, or any other fully resorbable biocompatible medium or a combination thereof, either in the form of injectable liquid, gel, putty or cement or in the form of mouldable liquid, cement, putty, gel, flexible sheets, mesh or sponge or other readily applicable method.

24. The drug of any one of claims 21 to 23 when further admixed with bone graft, bone graft substitutes or extenders or a combination thereof.

25. The drug of any one of claims 1 to 17 and 21 to 24 when used to treat a patient suffering from bone loss resulting from trauma including fracture, delayed union or non-union of bone, bone loss due to infection following open fracture or open treatment of a closed fracture, a bone defect unrelated to trauma including tumour or cyst formation, osteolysis from metabolic disorders such as hyperparathyroidism or renal failure, osteomyelitis, or surgical removal of bone.

26. The drug of any one of claims 1 to 17 and 21 to 24 when used to treat a patient suffering from bone defects in the maxilla, mandible or the skull.

27. The drug of any one claims 1 to 17 and 21 to 24 when used to treat a patient who has undergone an osteotomy and bone grafting is required to assist bony union.

28. The drug of any one of claims 1 to 17 and 21 to 24 when used to treat a patient suffering from congenital pseudarthrosis of the tibia, fibula or other bone, or a related condition or a patient who is undergoing revision or complicated primary joint arthroplasty, where bone grafting is required.

29. A drug selected from the group consisting of at least one bisphosphonate when used for treating a patient requiring a spinal or other joint arthrodesis.

30. The drug of claim 29 when used to increase the osteoinductive and/or osteoconductive potential of particles of graft obtained locally at a selected area during surgery.

31. The drug of claim 29 or claim 30 wherein the at least one bisphosphonate is administered to the patient parenterally at or near the time of the surgery.

32. The drug of claim 29 or claim 30 wherein the at least one bisphosphonate is admixed with bone graft, bone marrow, bone graft substitute or extender, or combinations thereof.

33. A drug selected from the group consisting of at least one bisphosphonate when used for improving the osteoinductive and/or osteoconductive potential of bone graft, bone graft substitutes or extenders wherein said bone graft, bone graft substitutes or extenders are held within a cage for interbody spinal fusion.

34. The drug of claim 33 wherein the cage is made from a material selected from titanium, any other biocompatible metal or alloy or a resorbable polymer or calcium containing complex.

35. The drug of claim 33 or claim 34 wherein the at least one bisphosphonate is administered systemically to a patient.

36. The drug of claim 33 wherein the at least one bisphosphonate is admixed with the bone graft, bone graft substitutes or extenders.

37. A method of performing a bone graft procedure including the step of administering to a patient a therapeutically effective amount of a drug selected from the group consisting of at least one bisphosphonate.

38. A method of performing bone grafting in a patient, said method including the steps of:
(a) harvesting autogenous bone graft from a donor site of the patient;
(b) delivering said harvested autogenous bone graft to a graft site;
(c) administering a drug selected from the group consisting of at least one bisphosphonate to the patient to increase the osteoinductive and/or osteoconductive potential of the autogenous bone graft.

39. A method of performing bone grafting in a patient, said method including the steps of:
(a) delivering a bone graft, bone graft substitute, an extender or a combination thereof to a graft site; and
(b) administering a drug selected from at least one bisphosphonate to the patient to increase the osteoinductive and/or osteoconductive potential of the bone graft, bone graft substitute or the extender.

40. The method of claim 38 wherein the at least one bisphosphonate is admixed locally at the graft site with the autogenous bone graft.

41. The method of claim 39 wherein the at least one bisphosphonate is admixed locally at the graft site with the bone graft, bone graft substitute or the extender or a combination thereof.

42. The method of claim 38 or claim 39 wherein the bisphosphonate is administered systemically to the patient either pre-operatively, intra-operatively or postoperatively.

43. The method of claim 39 wherein the at least one bisphosphonate is added to the bone graft, bone graft substitute or extender prior to implantation into the graft site.

44. A method of performing bone grafting in a patient, said method including the steps of:
(a) harvesting bone marrow from a patient;
(b) delivering said harvested bone marrow to a graft site;
(c) administering a drug selected from the group consisting of at least one bisphosphonate to the patient to increase the osteoinductive potential of the implanted bone marrow.

45. A device for performing a bone grafting procedure, the device including a first receiving means for receiving any one of bone graft, bone marrow, bone graft substitutes, extenders or carriers or a combination thereof and at least a second receiving means for receiving a drug selected from the group consisting of at least one bisphosphonate, the device further including a mixing means to allow mixing of said drug with said bone graft, bone marrow, bone graft substitutes, extenders, carriers or combination thereof.

46. The device of claim 45 further including a means to allow the mixture to be directly injected or implanted into a graft site of a patient.
